(19) United States
(12) Patent Application Publication
(10) Pub. No.: US 2024/0212865 A1
(43) Pub. Date: Jun. 27, 2024

(54) SKILL LEARNING FOR DYNAMIC TREATMENT REGIMES

(71) Applicant: NEC Laboratories America, Inc., Princeton, NJ (US)

(72) Inventors: Wenchao Yu, Plainsboro, NJ (US); Wei Cheng, Princeton Junction, NJ (US); Haifeng Chen, West Windsor, NJ (US)

(21) Appl. No.: 18/539,506

(22) Filed: Dec. 14, 2023

Related U.S. Application Data
(60) Provisional application No. 63/434,133, filed on Dec. 21, 2022.

(51) Int. Cl. G16H 50/70; G16H 10/60; G16H 20/00; G16H 50/20

(52) U.S. Cl. CPC G16H 50/70; G16H 10/60; G16H 20/00; G16H 50/20

(57) ABSTRACT

Methods and systems for training a healthcare treatment machine learning model include segmenting a patient trajectory, which includes a sequence of patient states and treatment actions. A machine learning model is trained based on segments of the patient trajectory, including a prototype layer that learns prototype vectors representing respective classes of trajectory segments and an imitation learning layer that learns a policy to select a treatment action based on an input state and a skill embedding.

### RELATED APPLICATION INFORMATION

This application claims priority to U.S. Patent No. 63/434,133, filed on Dec. 21, 2022, incorporated herein by reference in its entirety.

### BACKGROUND

### Technical Field

The present invention relates to machine learning systems and, more particularly, to systems for learning skills from historical records of medical treatments.

### Description of the Related Art

A dynamic treatment regime is a set of sequential treatment decision rules that can be used to provide suggestions for treating patients. While machine learning systems may be used to build a dynamic treatment regime from historical records, the underlying rationales for those historical actions may not be interpretable, which creates challenges in real clinical scenarios. Furthermore, a single policy may be insufficient to handle a patient's changing needs.

### SUMMARY

A method for training a healthcare treatment machine learning model includes segmenting a patient trajectory, which includes a sequence of patient states and treatment actions. A machine learning model is trained based on segments of the patient trajectory, including a prototype layer that learns prototype vectors representing respective classes of trajectory segments and an imitation learning layer that learns a policy to select a treatment action based on an input state and a skill embedding.

A system for training a healthcare treatment machine learning model includes a hardware processor and a memory that stores a computer program. When executed by the hardware processor, the computer program causes the hardware processor to segment a patient trajectory, which includes a sequence of patient states and treatment actions, and to train a machine learning model based on segments of the patient trajectory, including a prototype layer that learns prototype vectors representing respective classes of trajectory segments and an imitation learning layer that learns a policy to select a treatment action based on an input state and a skill embedding.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a healthcare facility where skill imitation is used to guide treatment, in accordance with an embodiment of the present invention;

FIG. 2 is a diagram of a patient with an automated treatment system, in accordance with an embodiment of the present invention;

FIG. 3 is a block diagram of a skill imitation model that can operate on segments of a patient trajectory, in accordance with an embodiment of the present invention;

FIG. 4 is a block/flow diagram of a method of training and using a skill imitation model for patient treatment, in accordance with an embodiment of the present invention;

FIG. 5 is a block/flow diagram of a method for monitoring and treating a patient using a skill imitation model, in accordance with an embodiment of the present invention;

FIG. 6 is a block diagram of a computer system that can train and use a skill imitation model for treating a patient, in accordance with an embodiment of the present invention;

FIG. 7 is a diagram of a neural network architecture that can be used as part of a skill imitation model, in accordance with an embodiment of the present invention; and

FIG. 8 is a diagram of a deep neural network architecture that can be used as part of a skill imitation model, in accordance with an embodiment of the present invention.

### DETAILED DESCRIPTION OF PREFERRED EMBODIMENTS

Imitation learning may be used to learn a mapping between states and actions when devising a treatment for a patient in a healthcare context. The imitation learning aims to replicate expert behavior, such as diagnosis and treatment actions performed by doctors, based on demonstrations from a set of records. To that end, an interpretable sequence modeling framework may be used to identify an expert's trajectory based on sequence data with temporal characteristics. Learning and inference may be performed at a segment level, which captures temporal variability of states and identifies skills that are transferable across different trajectories. This provides flexibility compared to a trajectory-level formulation, as trajectories can be represented by multiple segments. By aggregating the learned prototypes at each segment, the obtained skill serves as conditional information that implicitly guides a policy network to differentiate between patterns and to provide accurate treatment recommendations. These recommended treatments may furthermore be interpreted at the segment level by tracing back to the exemplar segments.

An interpretable skill learning model is therefore provided to learn treatment policies, exploiting the segment-level expert demonstrations and results in representative, transferable skills across different trajectories. The model learns to capture exemplar segments and constructs a faithful skill embedding for imitation learning tasks.

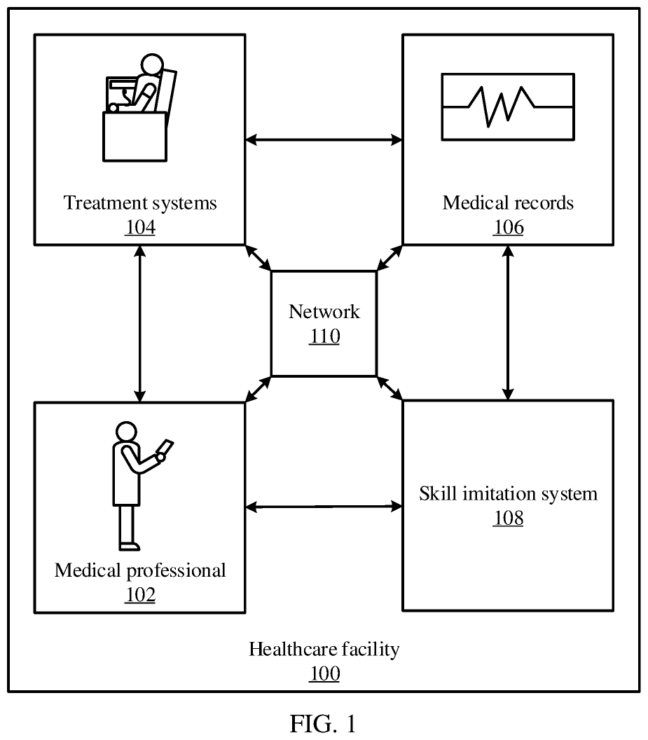

Referring now to FIG. 1, a diagram of skill learning is shown in the context of a healthcare facility 100. This may be used to help monitor and treat multiple patients, for example responding to changes in their particular healthcare needs. The healthcare facility may include one or more medical professionals 102 who provide information relating to events and measurements of system status to skill imitation system 108. Treatment systems 104 may furthermore monitor patient status to generate medical records 106 and may be designed to automatically administer and adjust treatments as needed.

Based on information drawn from at least the medical professionals 102, treatment systems 104, and medical records 106, skill imitation system 108 learns skills applied by the medical professionals 102 in response to developing patient conditions. For example, the medical records 106 may include historical patient healthcare conditions (e.g., biometric information and a description of symptoms) and actions taken by the medical professionals 102 responsive to those conditions.

The different elements of the healthcare facility 100 may communicate with one another via a network 110, for example using any appropriate wired or wireless communications protocol and medium. Thus the skill imitation system 108 may access remotely stored medical records 106, may communicate with the treatment systems 104, and may receive instructions and send reports to medical professionals 102. In particular, the skill imitation system 108 may automatically trigger treatment changes for a patient, responsive to new information gleaned from the medical records 106, by sending instructions to the treatment systems 104.

In some cases, the skill imitation system 108 may generate a specific treatment plan for the patient, including a prescription plan that includes drugs that will help treat the patient, a meal provision plan to address the patient's dietary needs, a rehabilitation plan to provide for physical therapy and other activities needed for the patient to recover, and a discharge destination plan that indicates whether the patient may return home, should remain, or should be transferred to another healthcare facility. The output of the skill imitation system 108 may therefore include one or a combination of the above automatic treatments and plan outputs.

Referring now to FIG. 2, patient 202 is shown in the context of a healthcare system. For example, the patient 202 may be in a hemodialysis (also known simply as “dialysis”) session. During dialysis, a dialysis machine 204 automatically draws the patient's blood, processes and purifies the blood, and then reintroduces the purified blood to the patient's body. Dialysis can take as long as four hours to complete, and may be performed every three days, though other durations and periods are contemplated. Although dialysis is specifically contemplated, it should be understood that any appropriate medical procedure or monitoring may be used instead.

Before, during, and after a dialysis session, a patient 202 may experience a health event relating to the treatment. Such health events can be dangerous to the patient 202, but can be predicted based on knowledge of previous health events and the patient's present health metrics. The recommendation 208 may furthermore include information relating to the type of event that is predicted, as well as measurements of the patient's status It is specifically contemplated that this recommendation may be made before the dialysis session begins, so that treatment can be adjusted.

The recommendation may be made based on a variety of input information. Part of that information includes a static profile of the patient, for example including information such as age, sex, starting time of dialysis, previous health events, etc. The information also includes dynamic data, such as dialysis measurement records, which may be taken at every dialysis session, blood pressure, weight, venous pressure, blood test measurements, and cardiothoracic ratio (CTR). The blood test measurements may be taken regularly, for example at a frequency of twice per month, and may measure such factors as albumin, glucose, and platelet count. The CTR may also be taken regularly, for example at a frequency of once per month. Dynamic information may also be recorded during the dialysis session, for example using sensors in the dialysis machine 204. The dynamic information may be modeled as time series over their respective frequencies.

In addition, the systems themselves may be monitored within a healthcare environment. For example, operational parameters of a dialysis machine 204 or any other system in a hospital or other healthcare facility many be monitored, along with a history of past events at the system, to predict events as described below.

During treatment, the status of the patient 202 may be continuously monitored, for example tracking the patient's heart rate and other vital signs. In the event that the patient's vital signs indicate an imminent or ongoing adverse health event, the treatment may be altered accordingly. For example, the treatment systems may automatically administer a drug or shut down treatment responsive to a negative health event.

To train the skill imitation system 108, the medical records 106 may include a set of doctors' demonstration trajectories, each of which has a sequence of state-action pairs (sₜ, aₜ), where sₜ denotes a given patient's state at a time t and aₜ indicates the treatment action taken by a medical professional 102. The skill imitation system 108 learns a policy that can replicate the treatment actions taken by the medical professionals 102. Imitation learning may be built upon step-level demonstrations, without consideration of the evolving patient symptoms and corresponding treatments.

The imitation learning task may instead be formulated at the level of a sequence of states, to exploit the sequential nature of the demonstrations. Each trajectory may thus be divided into segments, and imitation learning may be performed on respective segments. The skills at each segment may be representative and transferable across different trajectories. Each state in the segment comes with a historical state segment from the same trajectory, so that the dynamics of the patient's state and treatment demonstration can also be exploited at the segment level.

Given a set of segments

$${\{ {[ ( {s}_{t}^{( j )} , {a}_{t}^{( j )} ) ]}_{t = 1}^{m} \}}_{j = 1}^{n}$$

split from the original trajectories, where m is a fixed segment length and n is the number of segments, a set of prototypes may be determined that represent exemplar segments in the training data. The prototypes can be assembled as a skill embedding to facilitate the imitation learning of a dynamic treatment regime at each step t.

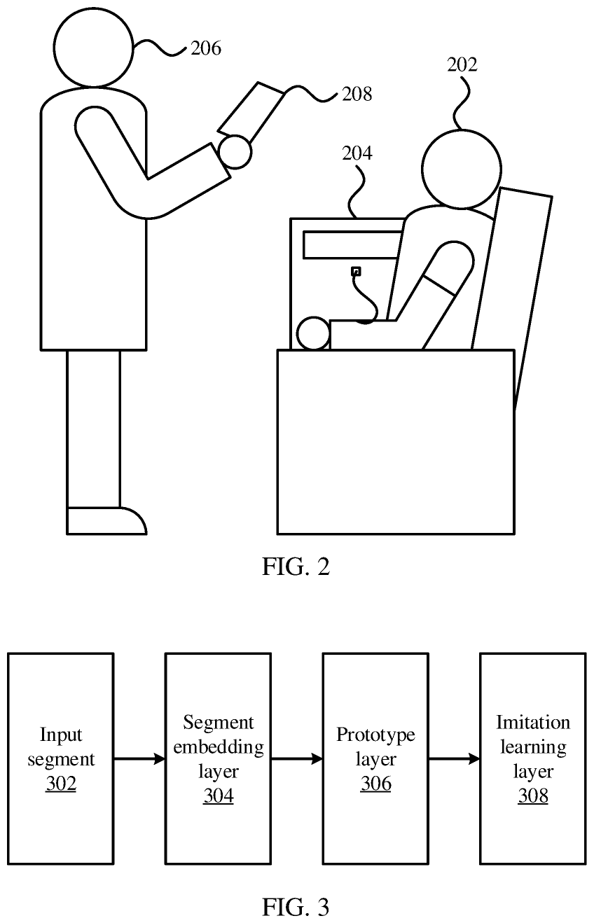

Referring now to FIG. 3, an imitation learning model is shown. The architecture of the model may include a segment embedding layer 304, a prototype layer 306, and an imitation learning layer 308. The subscript j, representing the order of an instance, is omitted herein for simplicity.

The input segment 302 associated with a patient's state at step t may be represented as [sₜ₋ₘ> . . . , sₜ₋₁, sₜ]∈ [symbol] ^{m×d}, where m denotes the length of the segment and d denotes the number of dimensions of the player's state. The segment embedding layer 304 extracts an embedding of the input segment 302. The segment embedding layer 304 may include a multilayer perceptron (MLP) with input neurons that accept respective elements of the input segment 302, followed by a one-dimensional convolutional layer that encodes the segment information. By sharing the same MLP encoder, the state embedding fₜ ∈ [symbol] ^{d′} at step t can be generated as fₜ=MLP(sₜ). The segment of encoded state in the embedding space [fₜ₋ₘ, . . . , fₜ₋₁, fₜ]∈ [symbol] ^{m×d′} is fed into the one-dimensional convolutional layer to generate segment embedding zₜ ∈ [symbol] ^{h×1}:

$${z}_{t} = {CONCAT}_{i = 0}^{h - 1} ( {W}_{i} ⋆ {f}_{t - m + 1 : t} + {b}_{i} )$$

where Wᵢ ∈ [symbol] ^{m×d′} denotes the i^{th} convolutional kernel out of h total kernels, bᵢ ∈ [symbol] denotes a corresponding bias term, the * operator provides a sum of row-wise cross-correlation, d′ is the dimensionality of the states after processing by the MLP encoder, and CONCAT provides the concatenation of all convolution results on h kernels.

The segment embedding layer may be implemented in other ways as well, for example using a long-short term memory (LSTM) or gated recurrent unit (GRU) architecture, or using a transformer architecture. However, as long segments may be uncommon in dynamic treatment regimes, the one-dimensional convolutional layer may be more efficient and effective in extracting the relevant embedding for short segments.

The prototype layer 306 may use k prototype vectors [p₁, . . . , pₖ] ∈ [symbol] ^{k×h}, which may be viewed as trainable model parameters and which have the same dimensionality as the segment embedding zₜ. The learning of prototypes has advantages for interpretability, as the original data segments on which the prototype vectors are projected may be retained for analysis.

Each prototype vector represents a class of exemplar segments reflecting the patient's state at one stage. The similarity score between the segment embedding zₜ and each prototype vector may be determined as Sim(zₜ, pᵢ)=e\hat{ }(−∥zₜ−pᵢ∥₂²), where pᵢ denotes the i^{th} prototype vector, ∥·∥₂ denotes the L2 norm, and the exponential function brings the similarity score to a bounded range for numerical stability.

The similarity scores of all prototype-embedding pairs are scaled between [0,1] and the resulting scared score for the i^{th} prototype-embedding pair is:

$$( {z}_{t} , {p}_{i} ) = \frac{{e}^{- {‖ {z}_{t} - {p}_{i} ‖}_{2}^{2}}}{{\sum }_{i = 1}^{k} {e}^{- {‖ {z}_{t} - {p}_{i} ‖}_{2}^{2}}}$$

All scaled scores form a weighting vector Wₚ ∈ [symbol] ^{k×1} as: Wₚ=[ [symbol] (zₜ, p₁), . . . , [symbol] (zₜ, pₖ)]. Based on Wₚ, the skill embedding oₜ ∈ [symbol] ^{1×h} can be constructed for the segment by the weighted combination of vectors p:

$${o}_{t} = {W}_{p}^{T} \cdot p$$

where the operator · denotes the inner product operation. Instead of the original segment embedding zₜ, all prototype vectors p are involved in the final skill embedding oₜ, so that the skill embedding can be interpreted based on the original state segments via prototype-segment association.

The imitation learning layer 308 modifies a flat policy network π_{θ}(aₜ|sₜ) that is parameterized by θ, learning mappings from sₜ to aₜ by incorporating the skill embedding oₜ that serves as a high-level indicator guiding the agent to mimic expert demonstrations sampled from the expert policy π_{E}(aₜ|sₜ). The patient state sₜ is concatenated with the skill embedding oₜ as conditional information and is fed to the contextual policy π_{θ}(aₜ|oₜ, sₜ), from which the primitive output action at for the dynamic treatment regime task can be derived:

$${a}_{t} ← {π}_{θ} ( {a}_{t} \vert {o}_{t} , {s}_{t} )$$

The contextual policy network may be built based on a behavior cloning model, which aims to imitate the doctor's medication at each time step t, by treating it as a supervised learning problem. The actual policy network may be implemented by a four-layer MLP. The interpretable skill learning explicitly models the segments of a patient's states with prototype vectors that are learned and regularized by behavior cloning and multiple interpretable learning objectives, based on which the user can obtain an explanation by tracing back to the training segments.

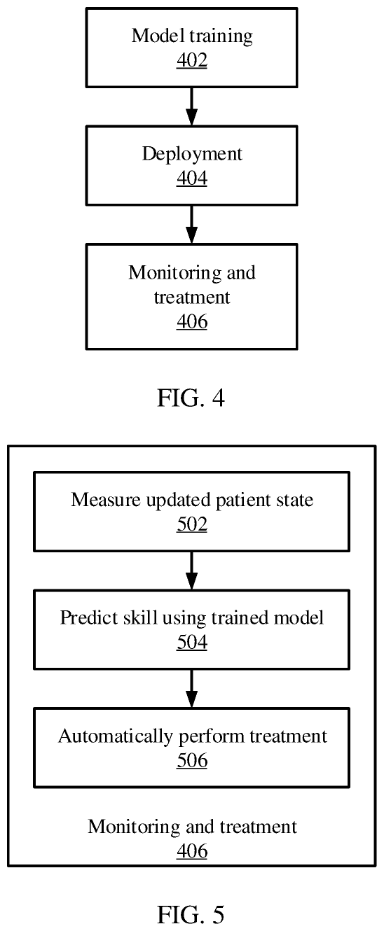

Referring now to FIG. 4, a method for training and using a skill imitation model is shown. Block 402 trains the model, using a set of historical examples of training that include sequences of patient states and treatment actions taken by medical professionals, making up trajectories. Once the model has been trained, it may be deployed 404 to a skill imitation system 108 at a healthcare facility 100. There, it may be used for monitoring and treatment 406 of patients, coordinating treatment actions by the treatment systems 104 and healthcare professionals 102.

During training 402, trajectories from the historical data are split into segments of a fixed size. Imitation learning is performed to train the policy, as described above, to minimize an objective function:

$$ℒ = {ℒ}_{IM} + {λ}_{1} {ℒ}_{cluster} + {λ}_{2} {ℒ}_{e v i d e n c e} + {λ}_{3} {ℒ}_{d i v e r s i t y}$$

where λ₁, λ₂, and λ₃ are weighting coefficients between zero and one. The objective function includes multiple terms, including an imitation learning term [symbol] _{IM}. Given a batch of segments with size n, the contextual policy aims to mimic the doctor's demonstration at the segment level in a supervised manner:

$${ℒ}_{IM} = {\sum }_{j = 1}^{n} {\sum }_{t = 1}^{m} {π}_{E} ( {a}_{t}^{( j )} \vert {s}_{t}^{( j )} ) log {π}_{θ} ( {a}_{t}^{( j )} \vert {o}_{t}^{( j )} , {s}_{t}^{( j )} )$$

where m is the length of the segment, and πE denotes the expert policy from which the demonstrations are sampled.

To improve the interpretability of the skill learning model, three regularizing components may be used for the learning of prototype vectors, including terms relating to the clustering structure of segment embedding, the segment-prototype evidence, and the diversity of prototypes. The clustering structure regularization enforces the segment embedding zₜ to be as close to the nearest prototype as possible:

$${ℒ}_{cluster} = {\sum }_{j = 1}^{n} {min}_{i ∈ [ k ]} {‖ {z}_{t}^{( j )} - {p}_{i} ‖}_{2}^{2}$$

where [k] denotes the integer set with the max element k representing all prototype vectors.

The prototype-segment evidence regularization imposes a dual optimization objective regarding segment embedding and prototype vectors. The regularization encourages each prototype vector to be as similar to a segment embedding as possible:

$${ℒ}_{e v i d e n c e} = {\sum }_{i = 1}^{k} {min}_{j ∈ [ n ]} {‖ {p}_{i} - {z}_{t}^{( j )} ‖}_{2}^{2}$$

The clustering structure and prototype-segment evidence terms interact with each other and jointly constrain the learning of both the segment embedding layer 308 and the prototype layer 306 toward a clear and interpretable structure.

Similarity between each pair of prototype vectors may also be penalized, as indistinguishable prototype vectors representing similar patients may be redundant, while encouraging the diversity of prototypes improves generalizability when encountering new segments and trajectories. A diversity regularization term may be imposed as follows:

$${ℒ}_{diversity} = {\sum }_{i = 1}^{k} {\sum }_{{i}^{′} \neq i}^{k} max ( 0 , {d}_{min} - {‖ {p}_{i} - {p}_{{i}^{′}} ‖}_{2}^{2} )$$

where dₘᵢₙ is a proximity threshold determining whether to penalize a given prototype pair. The above constraints are applied to the last step t=m within each input segment, as it includes the entire information of the segment, without padding the patient state/states from the previous segment in the same trajectory.

Training 402 continues until the training loss [symbol] converges, with the prototype vectors being optimized to be close to segment embeddings from the training data. However, the prototype vectors are not interpretable at this stage, as there is no correspondence between them and the actual segments. To induce the association between prototypes and segments in the training data, each prototype pᵢ may be assigned to its closest segment in the latent space:

$${p}_{i} ← arg {min}_{{z}_{t}^{( j )} ∈ {𝒵}_{t r a i n}} {‖ {p}_{i} - {z}_{t}^{( j )} ‖}_{2}^{2}$$

where [symbol] ₜᵣₐᵢₙ is a set of segment embeddings generated by feeding all segments of the training dataset to the segment embedding layer 304. The performance tends to increase with the number of prototypes, but stabilizes so that at some point the overhead of using additional prototypes is not justified. In some embodiments, k=25 prototypes may be sued.

Referring now to FIG. 5, additional detail on the monitoring and treatment 406 is shown. Block 502 measures an updated patient state, for example using treatment systems 104, adding the updated patient state information to the medical records 106. This information may include health indicators, such as heart rate, blood pressure, laboratory test results, as well as the patient's medication and treatment history. The collected data makes up the patient's state sₜ.

The new patient state may be input to the trained model in block 504, which generates a corresponding skill output. The trained model converts the state into an embedding, which is then used to identify the most appropriate prototype. The model's imitation learning is activated and generates action plans based on both the input state and the identified prototype. The action plans may include recommendations or treatment indications, for example specifying particular medication dosages. The output skill indicates a treatment to be performed, and block 506 automatically performs the treatment, for example by sending an instruction to the treatment system 502. The treatment may further be provided to medical professionals 102. This treatment information can assist the medical professionals 102 in decision-making for managing patients.

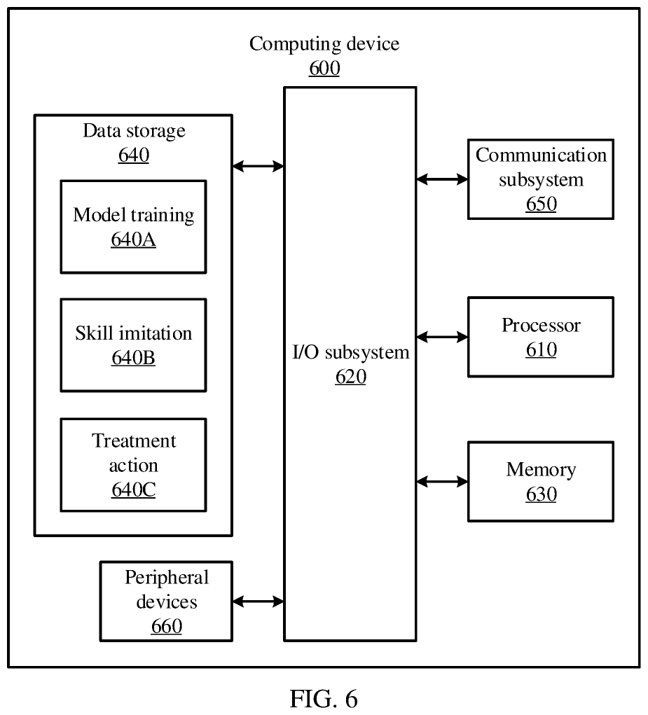

Referring now to FIG. 6, an exemplary computing device 600 is shown, in accordance with an embodiment of the present invention. The computing device 600 is configured to perform skill recognition.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for training a model, 640B for predicting an event, and/or 640C for performing a corrective action responsive to the predicted event. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

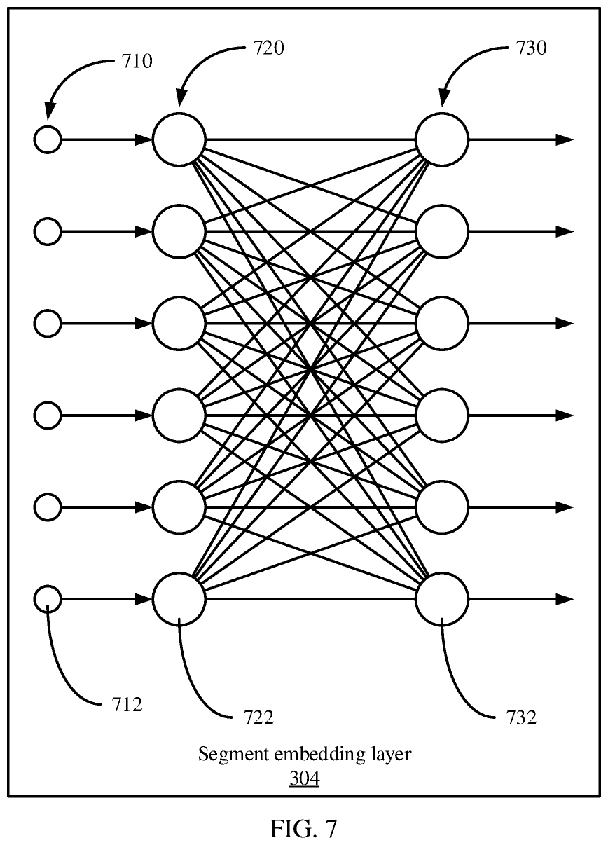

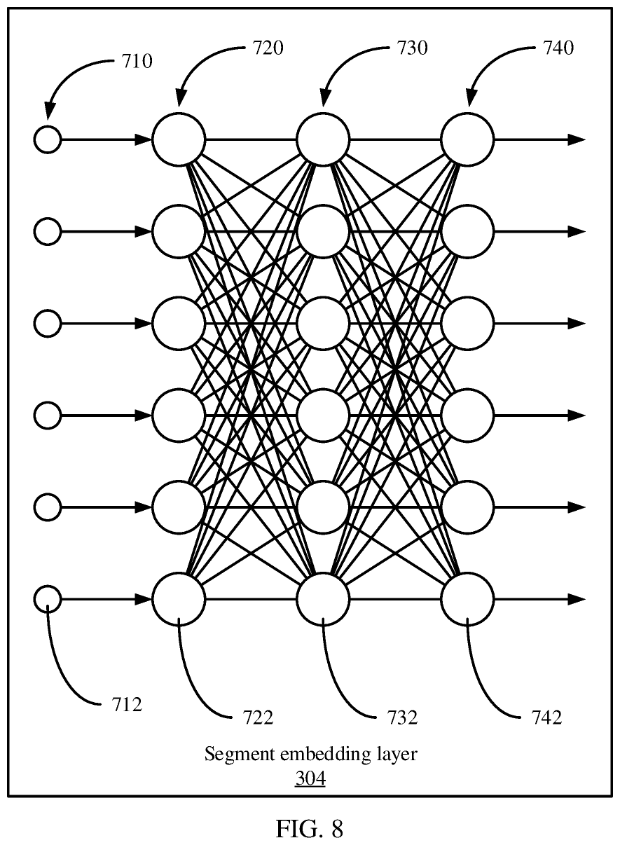

Referring now to FIGS. 7 and 8, exemplary neural network architectures are shown, which may be used to implement parts of the present models, such as the segment embedding layer 304. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for training a healthcare treatment machine learning model, comprising:
segmenting a patient trajectory, which includes a sequence of patient states and treatment actions; and
training a machine learning model based on segments of the patient trajectory, including a prototype layer that learns prototype vectors representing respective classes of trajectory segments and an imitation learning layer that learns a policy to select a treatment action based on an input state and a skill embedding.

2. The method of claim 1, further comprising embedding the segmented patient trajectory using a segment embedding layer of the machine learning model.

3. The method of claim 2, wherein the segment embedding layer includes a multilayer perceptron and a one-dimensional convolutional layer.

4. The method of claim 1, further comprising:
measuring a patient's state information;
selecting a treatment action based on a skill predicted by the trained model, based on the measured state information; and
notifying a medical professional of the treatment action to assist the medical professional in decision-making for patient management.

5. The method of claim 1, wherein the skill embedding includes a weighted combination of the prototype vectors based on how similar the prototype vectors are to the segmented patient trajectory.

6. The method of claim 1, wherein training the machine learning model includes minimizing a loss function that includes an imitation learning term, a clustering structure regularization term, a prototype segment evidence regularization term, and a diversity regularization term.

7. The method of claim 6, wherein the imitation learning term is expressed as:
$${ℒ}_{IM} = {\sum }_{j = 1}^{n} {\sum }_{t = 1}^{m} {π}_{E} ( {a}_{t}^{( j )} \vert {s}_{t}^{( j )} ) log {π}_{θ} ( {a}_{t}^{( j )} \vert {o}_{t}^{( j )} , {s}_{t}^{( j )} )$$
where m is a length of a segment, n is a number of segments, π_{E} is an expert policy, aₜ^{(j)} is an action performed at step t for segment j, sₜ^{(j)} is a patient state at step t for segment j, π_{θ} is a learned policy, and oₜ^{(j)} is a skill embedding at step t for segment j.

8. The method of claim 6, wherein the clustering structure regularization term is expressed as:
$${ℒ}_{cluster} = {\sum }_{j = 1}^{n} {min}_{i ∈ [ k ]} {‖ {z}_{t}^{( j )} - {p}_{i} ‖}_{2}^{2}$$
the prototype segment evidence regularization term is expressed as:
$${ℒ}_{e v i d e n c e} = {\sum }_{i = 1}^{k} {min}_{j ∈ [ n ]} {‖ {p}_{i} - {z}_{t}^{( j )} ‖}_{2}^{2}$$
and the diversity regularization term is expressed as:
$${ℒ}_{diversity} = {\sum }_{i = 1}^{k} {\sum }_{{i}^{′} \neq i}^{k} max ( 0 , {d}_{min} - {‖ {p}_{i} - {p}_{{i}^{′}} ‖}_{2}^{2} )$$
where n is a number of segments, k is a number of prototype vectors, pᵢ is an i^{th} prototype vector, zₜ^{(j)} is a segment embedding at step t for segment j, and dₘᵢₙ is a proximity threshold.

9. The method of claim 1, wherein the treatment action includes at least one of a prescription plan, a meal provision plan, a rehabilitation plan, and a discharge destination plan.

10. The method of claim 1, wherein the treatment action includes an instruction to a treatment system to automatically administer a treatment to a patient.

11. A system for training a healthcare treatment machine learning model, comprising:
a hardware processor; and
a memory that stores a computer program which, when executed by the hardware processor, causes the hardware processor to:
segment a patient trajectory, which includes a sequence of patient states and treatment actions; and
train a machine learning model based on segments of the patient trajectory, including a prototype layer that learns prototype vectors representing respective classes of trajectory segments and an imitation learning layer that learns a policy to select a treatment action based on an input state and a skill embedding.

12. The system of claim 11, wherein the computer program further causes the hardware processor to embed the segmented patient trajectory using a segment embedding layer of the machine learning model.

13. The system of claim 12, wherein the segment embedding layer includes a multilayer perceptron and a one-dimensional convolutional layer.

14. The system of claim 11, wherein the prototype layer determines a similarity between the segments and the prototype vectors.

15. The system of claim 11, wherein the skill embedding includes a weighted combination of the prototype vectors based on how similar the prototype vectors are to the segmented patient trajectory.

16. The system of claim 11, wherein the computer program further causes the hardware processor to minimize a loss function that includes an imitation learning term, a clustering structure regularization term, a prototype segment evidence regularization term, and a diversity regularization term.

17. The system of claim 16, wherein the imitation learning term is expressed as:
$${ℒ}_{IM} = {\sum }_{j = 1}^{n} {\sum }_{t = 1}^{m} {π}_{E} ( {a}_{t}^{( j )} \vert {s}_{t}^{( j )} ) log {π}_{θ} ( {a}_{t}^{( j )} \vert {o}_{t}^{( j )} , {s}_{t}^{( j )} )$$
where m is a length of a segment, n is a number of segments, π_{E} is an expert policy, aₜ^{(j)} is an action performed at step t for segment j, sₜ^{(j)} is a patient state at step t for segment j, π_{θ} is a learned policy, and oₜ^{(j)} is a skill embedding at step t for segment j.

18. The system of claim 16, wherein the clustering structure regularization term is expressed as:
$${ℒ}_{cluster} = {\sum }_{j = 1}^{n} {min}_{i ∈ [ k ]} {‖ {z}_{t}^{( j )} - {p}_{i} ‖}_{2}^{2}$$
the prototype segment evidence regularization term is expressed as:
$${ℒ}_{e v i d e n c e} = {\sum }_{i = 1}^{k} {min}_{j ∈ [ n ]} {‖ {p}_{i} - {z}_{t}^{( j )} ‖}_{2}^{2}$$
and the diversity regularization term is expressed as:
$${ℒ}_{diversity} = {\sum }_{i = 1}^{k} {\sum }_{{i}^{′} \neq i}^{k} max ( 0 , {d}_{min} - {‖ {p}_{i} - {p}_{{i}^{′}} ‖}_{2}^{2} )$$
where n is a number of segments, k is a number of prototype vectors, pᵢ is an i^{th} prototype vector, zₜ^{(j)} is a segment embedding at step t for segment j, and dₘᵢₙ is a proximity threshold.

19. The system of claim 11, wherein the treatment action includes at least one of a prescription plan, a meal provision plan, a rehabilitation plan, and a discharge destination plan.

20. The system of claim 11, wherein the treatment action includes an instruction to a treatment system to automatically administer a treatment to a patient.
